(19) United States
(12) Patent Application Publication
(10) Pub. No.: US 2021/0268040 A1
(43) Pub. Date: Sep. 2, 2021

(54) STREPTOCOCCUS THERMOPHILUS CNRZ160 STRAIN FOR THE TREATMENT AND PREVENTION OF INTESTINAL INFLAMMATION AND ASSOCIATED DISORDERS IN AN INDIVIDUAL

(71) Applicants: INSTITUT NATIONAL DE RECHERCHE POUR L'AGRICULTURE L'ALIMENTATION ET L'ENVIRONNEMENT, Paris (FR); UNIVERSITE DE LORRAINE, Nancy (FR); UNIVERSITE CLERMONT AUVERGNE, Clermont-Ferrand (FR)

(72) Inventors: Dominique DARDEVET, Perignat lès Sarlieve (FR); Isabelle AUZELOUX, Clermont Ferrand (FR); Marianne JARZAGUET, Loubeyrat (FR); Jérémie DAVID, Beaumont (FR); Jean-Marc CHATEL, Meudon (FR); Annie DARY-MOUROT, Bicqueley (FR); Claire CHERBUY, Les Ulis (FR); Philippe LANGELLA, Vélizy-Villacoublay (FR)

(21) Appl. No.: 17/261,727

(22) PCT Filed: Jun. 19, 2019
(86) PCT No.: PCT/EP2019/066178

§ 371 Date: Jan. 20, 2021

(30) Foreign Application Priority Data
Jul. 26, 2018 (FR) 1856950

(51) Int. Cl. A61K 35/744; A61P 1/00; A61P 29/00; A61K 9/00; A23L 33/00; A23L 33/135; A23L 2/52; A23C 9/123

(52) U.S. Cl. CPC A61K 35/744; A61P 1/00; A61P 29/00; A61K 9/0056; A61K 9/0095; A23V 2002/00; A23L 33/135; A23L 2/52; A23C 9/1238; A23Y 2240/75; A23L 33/40

(57) ABSTRACT

The present invention relates to a bacterial of the Streptococcus thermophilus species with reference CNRZ160, for use in the prevention and/or treatment of intestinal inflammation in an individual.

### FIELD OF THE INVENTION

The present invention relates to a strain of Streptococcus thermophilus with reference CNRZ160 for use in the prevention and/or treatment of intestinal inflammation, and of disorders caused by said intestinal inflammation, in an individual.

The invention relates more particularly to a strain of Streptococcus thermophilus with reference CNRZ160 for use in the prevention and/or treatment of loss of lean body mass.

### PRIOR ART

Inflammation is a natural biological process, which forms a normal part of the response to lesions or infections and helps to protect the organism against internal or external aggressive factors.

However, a dysfunction of the mechanisms of inflammation, in particular inflammation that is persistent or too abundant, may cause painful diseases and put the patient's life in danger. Such diseases comprise, for example, skin disorders, intestinal disorders, neurological disorders, arthritis and autoimmune diseases. Several of these inflammatory diseases are still without treatment or without adequate treatment.

Such an inflammation may affect all or part of the intestine. Thus, we may notably distinguish, in cases when inflammation is localized at the level of a particular region, enteritis (inflammation of the small intestine), duodenitis (inflammation of the duodenum), ileitis (inflammation of the ileum), jejunitis (inflammation of the jejunum), typhlitis (inflammation of the cecum), appendicitis (inflammation of the appendix) and colitis (inflammation of the colon). When several regions of the intestine are inflamed, we also use the terms enterocolitis (inflammation of the small intestine and colon), gastroenteritis (inflammation of the stomach and small intestine), hemorrhagic rectocolitis (also called ulcerative colitis, a chronic inflammatory disease of the intestines which more specifically affects the colon and the rectum) and Crohn's disease (chronic inflammatory disease of the intestines which affects the whole intestine and may extend for example to the oral cavity, the rectum or the anal canal).

Consequently, the investigation of and search for new strategies of anti-inflammatory treatment constitute a major topic in medicine and in biomedical research.

There is thus a constant need for new substances, in particular for probiotics, or compositions, for treating and/or preventing intestinal inflammation.

An individual suffering from or who has suffered from an intestinal inflammation may, at the same time as or after said inflammation, present disorders that may be caused by this inflammation, namely:
weight loss;
loss of lean mass;
muscle loss;
hypertrophy of the colon; and/or
alteration of intestinal permeability.

Consequently, the investigation and search for new strategies for preventing and treating weight loss, loss of lean mass, muscle loss, hypertrophy of the colon and/or a change in intestinal permeability also constitute a major topic in medicine and in biomedical research. There is thus also a constant need for new substances, in particular probiotics, or compositions, for the treatment and/or prevention of weight loss, loss of lean mass, muscle loss, hypertrophy of the colon and/or of a change in intestinal permeability, in particular in an individual with intestinal inflammation, and notably for the treatment and/or prevention of muscle loss.

### SUMMARY OF THE INVENTION

The aim of the present invention is to describe a new substance, in particular a probiotic, and compositions comprising same, for treating and/or preventing an inflammatory intestinal disorder and/or a disorder caused by said inflammatory intestinal disorder in an individual.

In the context of the present invention, the terms “prevent” and “prevention” denote the reduction, to a lesser degree, of the risk or probability of occurrence of a given phenomenon, i.e., in the present invention, of an inflammatory intestinal disorder and/or of a disorder caused by said inflammatory intestinal disorder in an individual.

In the context of the present application, the terms “treat” and “treatment” associated with an inflammatory intestinal disorder and/or a disorder caused by said inflammatory intestinal disorder according to the invention, denote a decrease, or even an interruption of said inflammatory intestinal disorder and/or of said disorder caused by this inflammatory intestinal disorder. Thus, for example, in the case of treatment of loss of muscle mass, these terms denote a decrease, or even an interruption, of the loss of muscle mass, or even an increase in muscle mass, this increase being achieved by an increase in the quantity of muscle fibers at the level of the muscle or muscles in question.

The present invention is based on the discovery of the properties of the strain of Streptococcus thermophilus with reference CNRZ160, deposited with the National Collection of Cultures of Microorganisms (CNCM) (Institut Pasteur, 25-28, rue du Docteur Roux 75724 Paris Cedex 15) under accession number CNCM 1-5334 Jul. 4, 2018, for the treatment and prevention, in an individual, both of intestinal inflammation but also of disorders caused by said inflammatory intestinal disorder.

According to the inventors' experimental results, a specific strain of Streptococcus thermophilus with reference CNRZ160 in fact possesses the unexpected capacity to restore intestinal permeability in vivo in individuals with intestinal inflammation.

Similarly, the inventors' experimental results demonstrate that a specific strain of Streptococcus thermophilus with reference CNRZ160 also possesses the unexpected capacity to reduce weight loss, loss of muscle mass and loss of lean mass in vivo in individuals with intestinal inflammation.

Moreover, other results demonstrate the capacity of a specific strain of Streptococcus thermophilus with reference CNRZ160 to limit, or even cancel, hypertrophy of the colon in individuals with intestinal inflammation.

Furthermore, the inventors' experimental results also demonstrate a return of protein synthesis to normal, both at the level of the colon and of the muscles, after treatment with a specific strain of Streptococcus thermophilus with reference CNRZ160.

As illustrated in the examples, the inventors have shown that the various properties of the strain CNRZ160 are not generally encountered in Streptococcus thermophilus. For example, the strain of Streptococcus thermophilus PBSMJ, deposited with the CNCM under accession number CNCM 1-5336 on Jul. 18, 2018, does not possess this advantageous property.

Finally, in addition, the present results demonstrate absence of mortality in individuals with intestinal inflammation who have been treated using a specific strain of Streptococcus thermophilus with reference CNRZ160. This property is also not generally encountered in Streptococcus thermophilus, since on the contrary, the administration of a strain of Streptococcus thermophilus PB5MJ leads to a very significant increase in the mortality of individuals with intestinal inflammation.

Thus, the present invention relates firstly to a bacterial strain of the species Streptococcus thermophilus deposited with the CNCM under accession number CNCM 1-5334, for use in the prevention and/or treatment of an inflammatory intestinal disorder and/or of a disorder caused by this inflammatory intestinal disorder, in an individual.

This strain identified by the inventors is therefore a probiotic strain which may be used for the applications indicated above.

This strain may be used in the sense of the invention in a live, semi-active, inactivated or dead form.

In the sense of the invention, a microorganism in a semi-active form is a microorganism whose capacity to proliferate is reduced, temporarily or permanently.

Thus, in the sense of the invention, an “inactivated” microorganism is a microorganism that is no longer capable of proliferating, temporarily or permanently.

In the sense of the invention, a “dead” microorganism is a microorganism that is permanently no longer capable of proliferating.

Dead or inactivated microorganisms may have intact or broken cell membranes. Thus, the term “inactivated” also denotes the extracts and lysates of microorganisms obtained as detailed above. Dead or inactivated microorganisms may be obtained by any method known by a person skilled in the art.

An inactivated probiotic microorganism suitable for the invention may be prepared by irradiation, thermal inactivation or lyophilization of a preparation of microorganism. These methods are known by a person skilled in the art.

More particularly, inactivation of probiotic microorganisms by irradiation may comprise the use of gamma rays, X-rays or exposure to UV. The type of radiation, intensity, dose and exposure times are adjusted by a person skilled in the art according to the quantity and the nature of the probiotic microorganisms to be inactivated.

Inactivation by lyophilization may be carried out by any method known in this field. Advantageously, probiotic microorganisms inactivated by lyophilization may be put back in culture.

A supplementary probiotic microorganism according to the invention may be used in whole form, i.e. essentially in its native form, or in the form of extracts or lysates comprising fractions and/or metabolites of this microorganism. Such a lysate may notably be prepared as indicated hereunder.

A lysate according to the invention may comprise some or all of the fractionated elements and/or of the metabolites resulting from lysis of the probiotic microorganism.

A lysate in the sense of the invention denotes the product obtained as a result of destruction or dissolution of biological cells by a phenomenon of cellular lysis causing release of the intracellular biological constituents contained naturally in the cells of the microorganism in question.

In the sense of the present invention, the term “lysate” is used indiscriminately for denoting the whole of the lysate obtained by lysis of the microorganism in question or only a fraction of the latter.

In the sense of the present invention, the term “whole lysate” is used to denote more precisely the whole of the lysate obtained by lysis of the microorganism in question.

The lysate used is therefore formed wholly or partly from the intracellular biological constituents and the constituents of the walls and cell membranes.

According to one embodiment, a lysate used for the invention is the whole of the lysate obtained by lysis of the microorganism in question.

This cellular lysis may be carried out using various technologies, such as for example thermal shock, with ultrasound, osmotic shock, or under mechanical stress, such as by centrifugation.

An individual according to the invention is preferably a mammal, including a nonhuman mammal, and is, in particular, a human.

According to a particular embodiment, said disorder caused by this inflammatory intestinal disorder is selected from the list consisting of weight loss, loss of lean mass, muscle loss, hypertrophy of the colon, alteration of intestinal permeability and any combination of these disorders. More particularly, said disorder caused by this inflammatory intestinal disorder is loss of lean mass and muscle mass.

Said colic inflammatory intestinal disorder may, in particular, be a chronic inflammatory disease of the intestine, and may more particularly be selected from the group consisting of Crohn's disease, hemorrhagic rectocolitis and pouchitis, in particular Crohn's disease and hemorrhagic rectocolitis.

According to another embodiment, the bacterial strain for the use according to the invention is included in a composition comprising a physiologically acceptable medium, preferably in an oral composition.

The term “physiologically acceptable medium” denotes a medium that is compatible with the organism of the individual to whom said composition is to be administered. It may be, for example, a nontoxic solvent such as water. In particular, said medium is compatible with oral administration.

A composition of the invention is preferably for the oral route.

According to one embodiment, the composition is suitable for administration of a daily dose representing from 10⁷ to 10¹¹ colony forming units (CFU), notably as a medicinal product, preferably in the form of a daily dose equivalent to 10⁹ CFU.

A composition of the invention for administration by the oral route may be selected from the group consisting of a food product, a drink, a pharmaceutical product, a nutraceutical, a food additive, a food supplement and a milk product and is preferably a milk product or a food supplement.

### LEGENDS OF THE FIGURES

FIG. 1 illustrates the intestinal permeability of different groups of mice (n=8/group) tube-fed, or not, with the strains PB5MJ or CNRZ160, a low-grade inflammation having been induced in these animals by the administration of DNBS. Intestinal permeability is measured by assay of FITC in the blood. The higher the concentration, the higher the intestinal permeability.

The groups of mice are as follows, from left to right: PBS ethanol: group of mice treated with PBS and with ethanol (uninflamed control); PBS DNBS: group of mice treated with DNBS and with PBS (inflamed control); PB5MJ DNBS: group of mice treated with DNBS and supplemented with Streptococcus thermophilus PB5MJ; CNRZ160 DNBS: animals treated with DNBS and supplemented with Streptococcus thermophilus CNRZ160.

Abscissa: groups of animals tested.

Ordinate: amount of FITC in the blood (μg/mL).

FIG. 2 illustrates the percentage of weight lost by different groups of mice relative to their weight before injection of DNBS triggering acute intestinal inflammation.

The groups of mice are as follows: PBS: group of mice treated with PBS (uninflamed control); Milk—DNBS: group of mice treated with DNBS and with Milk (inflamed control); CNRZ160—DNBS: animals treated with DNBS and supplemented with Streptococcus thermophilus CNRZ160.

Abscissa: number of days post-injection of DNBS.

Ordinate: Percentage of body weight remaining relative to the body weight at t0 (i.e. on the day of injection of DNBS).

FIG. 3 illustrates the mass of the gastrocnemius muscle of different groups of animals: DSS=group of animals treated with dextran sodium sulfate (DSS) for 28 days; Pair Fed=animals pair-fed relative to the group DSS; DSS+CNRZ160=animals treated with DSS for 28 days and supplemented with Streptococcus thermophilus CNRZ160; DSS+PB2=animals treated with DSS for 28 days and supplemented with Streptococcus thermophilus PB5MJ, as well as a control corresponding to a group of animals euthanized at the start of the experiments to measure the weight of the rats' muscles before treatment with DSS (Controls T0). Each bar having a letter different from another is significantly different. Thus, the bars indicating a, b or c are statistically different from one another. Bar a is statistically different from bars b, bc and c. Bar ab is statistically different from bar c. Bar c is statistically different from bars a, b and ab. Bar b is statistically different from bars a and c. Bar bc is statistically different from bar a.

Abscissa: groups of animals tested.

Ordinate: Mass of the gastrocnemius muscle in grams relative to the control group Controls T0.

FIG. 4 illustrates the percentage survival of different groups of mice at the end of 17 days post-administration of DSS.

The groups are as follows: PBS DSS: group of mice treated with DSS and with PBS (inflamed control); PB5MJ DSS: group of mice treated with DSS and supplemented with Streptococcus thermophilus PB5MJ; CNRZ160 DSS: animals treated with DSS and supplemented with Streptococcus thermophilus CNRZ160.

Abscissa: number of days post-injection of DSS.

Ordinate: percentage survival of the treated mice.

FIG. 5 illustrates the cumulative lean mass lost by different groups of rats in the days post-administration of DSS.

The groups are as follows: DSS: group of rats treated with DSS (inflamed control); Pair Fed=animals pair-fed relative to the group DSS; DSS CNRZ160: animals treated with DSS and supplemented with Streptococcus thermophilus CNRZ160.

Abscissa: number of days post-injection of DSS.

Ordinate: Cumulative loss of lean mass (g).

FIG. 6 illustrates the mass of the colon of different groups of rats after 28 days of administration of DSS.

The groups are as follows, from left to right: Controls T0: group of animals euthanized at the start of the experiments to measure the weight of the rats' muscles before treatment with DSS; Pair Fed=animals pair-fed relative to the group DSS; DSS: group of rats treated with DSS (inflamed control); DSS+CNRZ160: animals treated with DSS and supplemented with Streptococcus thermophilus CNRZ160.

Each bar having a letter different from another is significantly different. Thus, bars a are statistically different from bar b. Bar ab is not statistically different from bars a and b.

Abscissa: groups of animals tested.

Ordinate: Mass of the colon (g).

FIG. 7 illustrates the cumulative synthesis of colon proteins in mg per day for different groups of rats (from left to right): Pair Fed=animals pair-fed relative to the group DSS; DSS=group of animals treated with dextran sodium sulfate (DSS) for 28 days; DSS+CNRZ160=animals treated with DSS for 28 days and supplemented with Streptococcus thermophilus CNRZ160.

Each bar having a letter different from another is significantly different. Thus, bars a are statistically different from bar b.

Abscissa: groups of animals tested.

Ordinate: Quantity of proteins synthesized in the colon per day (mg/day).

FIG. 8 illustrates the cumulative synthesis of proteins of the gastrocnemius muscle in mg per day for different groups of rats (from left to right): Pair Fed=animals pair-fed relative to the group DSS; DSS=group of animals treated with dextran sodium sulfate (DSS) for 28 days; DSS+CNRZ160=animals treated with DSS for 28 days and supplemented with Streptococcus thermophilus CNRZ160; DSS+PB5MJ=animals treated with DSS for 28 days and supplemented with Streptococcus thermophilus PB5MJ.

Each bar having a letter different from another is significantly different. Thus, bars a are statistically different from bars b.

Abscissa: groups of animals tested.

Ordinate: Quantity of proteins synthesized in the muscle per day (mg/day).

### DETAILED DESCRIPTION OF THE INVENTION

The present inventors have undertaken extensive work to identify the capacity of a specific strain of Streptococcus thermophilus with reference CNRZ160 for treating and/or preventing an inflammatory intestinal disorder and/or a disorder caused by said inflammatory intestinal disorder, in an individual, in particular the disorders selected from the list consisting of weight loss, loss of lean mass, muscle loss, hypertrophy of the colon, alteration of intestinal permeability and any combination of these disorders, and notably weight loss in striated muscles.

Amyotrophy, also called myatrophy or muscular atrophy, corresponds to a decrease in the total number of muscle fibers, and more particularly of type II fibers, in an individual, and thus weakening of this individual.

This muscle wasting may develop more or less rapidly over time, may be localized or general, and may have various causes.

Thus, muscular atrophy is observed in individuals whose body is partially or completely immobilized, whether following application of a plaster cast for a fracture or during prolonged bedrest. This muscle wasting mainly results from lack of muscular activity and remains benign and reversible in young individuals without any chronic pathology.

In certain cases, this amyotrophy is associated with a myopathy of hereditary origin, among which we may mention Duchenne myopathy, facioscapulohumeral myopathy or Steiner disease.

It may also be an acquired amyotrophy, resulting either from inflammation, including for example an intestinal inflammation and/or an inflammation of the muscles (which is then called polymyositis), or from treatment having said muscle loss as a side effect. Thus, it has been observed that administration of cortisone at high dose and for a long time may be the cause of amyotrophy of this kind.

Certain amyotrophies have a neurological origin, as is the case for example in Charcot disease, spinal amyotrophy, poliomyelitis or nerve lesions.

Finally it may be age-related. In fact, age-related muscular dystrophy, also called sarcopenia, is a pathology defined by consensus by the European group EWGSOP (European Working Group on Sarcopenia in Older People—Cruz-Jentoft A J et al.; Age Ageing, 2010 July; 39(4): 412-423), and results in a high, progressive loss of muscle mass, strength and function with aging. This degeneration begins from the age of 30 years and may represent a progressive loss of the order of 3 to 8% of muscle mass per decade, with an acceleration of this degeneration starting from 50 years of age. Moreover, this decline of muscle mass is concomitant with an increase in fat mass.

In all cases, this loss of muscle mass may lead to a partial or total loss of autonomy of the individuals concerned, resulting in difficulties in performing the activities of everyday life, possibly accompanied by disorders of maintaining posture, disorders of gait, disorders of balance, a decrease in bone mass, increased fatigability, disorders of cardiovascular capacities and an increased risk of falling and therefore of fractures, which may then lead to forced immobilization, which would make the situation even worse.

Strain of Streptococcus thermophilus CNRZ160 of the Invention

This strain was deposited with the CNCM under accession number CNCM 1-5334 on Jul. 4, 2018.

Streptococcus thermophilus is a lactic acid and food bacterium and a thermophile, only present in the fermentation of milk, where it is responsible for the acidification of milk in the manufacture of yoghurt. This bacterium is also present in pressed hard cheeses cooked at high temperature.

It has been demonstrated in the past that the administration of specific bacteria, including Streptococcus thermophilus of reference FP4, resulted in a decrease of the decline of performance and muscle tension in the days following physical exercise causing damage to the muscles (see JAGER Ralf et al.; Nutrients 2016, 8, 642).

Moreover, AU2015100928 describes a probiotic combination comprising at least 2 bacteria selected from a list with 19 members, among which Streptococcus thermophilus is mentioned in a general way, for treating or preventing fatigue, promoting synthesis of ATP, stimulating/increasing energy levels, treating or preventing muscle pains, muscle fatigue and/or muscle degeneration or for promoting muscle repair. However, these are only unsubstantiated claims: the experimental section of this application does not comprise any element supporting such properties for this combination of probiotics. Moreover, as is demonstrated in the examples, the inventors have at present proved that the properties particularly considered for the present invention are not possessed by all the bacteria of the genus S. thermophilus.

Thus, the inventors have demonstrated that the aforementioned properties of the S. thermophilus strain CNRZ160 cannot be ascribed to the species S. thermophilus, since the existence of this property is unforeseeable for a given strain of S. thermophilus. In fact, these activities are illustrated in the examples as indicated above, in which a comparative test was conducted with a strain of S. thermophilus not forming part of the invention, namely the reference strain PB5MJ, which does not possess the properties of the CNRZ160 strain.

A suitable daily dose of a bacterial strain according to the invention is from 10⁷ to 10¹¹ colony forming units (CFU) as medicinal product, for example in the form of a daily dose equivalent to 10⁹ CFU.

A bacterium according to the invention is a probiotic bacterium. A probiotic bacterium according to the invention denotes a microorganism which, when ingested, exerts beneficial effects on human health. The latter may advantageously be ingested in a live form.

The bacterial strain of the invention can be administered to an individual in various ways, namely by the oral or rectal route. A bacterium according to the invention is preferably administered by the oral route.

According to a preferred embodiment, the bacterial strain of the invention is included in a composition comprising a physiologically acceptable medium. Such a composition is preferably for administration by the oral route, and in particular in the form of a food supplement.

Compositions

The present invention further relates to a composition comprising, in a physiologically acceptable medium, at least the bacterial strain Streptococcus thermophilus with reference CNRZ160.

A composition of the invention is preferably an oral or rectal composition, more preferably an oral composition.

According to one embodiment, a composition of the invention is an oral composition, i.e. it is intended for oral administration to a subject.

Such a composition may be in the form of a suspension, a tablet, a pill, a capsule, granules or a powder.

The composition according to the invention for administration by the oral route may be selected from the group consisting of a food product, a drink, a pharmaceutical product, a nutraceutical, a food additive, a food supplement or a milk product, and is, in particular, a milk product or a food supplement.

According to a preferred embodiment, a composition according to the invention is a milk product.

A milk product for oral administration according to the invention may be selected from the list consisting of a yoghurt, a whey, butter, cream, whole milk, partially skimmed milk, fully skimmed milk, a cheese, in particular a cooked cheese, in particular hard cheeses cooked at a temperature greater than or equal to 45° C., such as for example Emmental, Comte or Parmesan.

According to a preferred embodiment, a composition according to the invention is a food supplement.

A food supplement for oral administration may be present in hard capsules, soft capsules, tablets, sugar-coated tablets, pills, pastes, pastilles, gums, solutions or oral emulsions, a sirup or a gel.

Advantageously, a composition according to the invention, intended for oral administration, may be provided with a coating resistant to the gastric juice, in order to ensure that the bacterial strain of the invention included in said composition can pass through the stomach without being damaged. The bacterial strain may thus be released for the first time in the upper intestinal tract.

A food supplement according to the invention may further comprise a sweetener, a stabilizer, an antioxidant, an additive, a flavoring agent and/or a colorant.

Formulation of the latter is carried out by the usual methods for producing sugar-coated tablets, hard capsules, gels, hydrogels for controlled release, emulsions, tablets or capsules.

In another embodiment of the invention, a composition containing the bacterial strain of the invention is administered by the intrarectal route.

Preferably, rectal administration is in the form of a suppository, an enema or a foam.

In particular, a composition of the invention is suitable for administration of a daily dose representing from 10⁷ to 10¹¹ colony forming units (CFU) as a medicinal product, preferably a daily dose equivalent to 10⁹ CFU.

For example, a composition according to the invention may be administered to an individual who needs it, at a single daily dose of 1 g containing the bacterial strain S. thermophilus CNRZ160 of the invention in an amount equivalent to a dose between 10⁷ and 10¹¹ CFU, preferably 10⁹ CFU.

In another example, a composition according to the invention may be administered to an individual who needs it, at a single daily dose of 0.2 g containing the bacterial strain S. thermophilus CNRZ160 of the invention in an amount equivalent to an amount between 10⁷ and 10¹¹ CFU, preferably 10⁹ CFU.

In another example, a composition according to the invention may be administered to an individual who needs it, twice a day on the basis of two doses of 1 g, each dose containing, independently, the bacterial strain S. thermophilus CNRZ160 of the invention in an amount equivalent to an amount between 5·10⁶ and 5·10¹⁰ CFU (based on the dry weight), preferably 5·10⁸ CFU, so that the total daily dose of bacterial strain S. thermophilus CNRZ160 of the invention administered to the individual is as indicated above.

A composition according to the invention may further comprise at least one from: antioxidants, fish oils, DHA, EPA, vitamins, minerals, phytonutrients, a protein, a lipid, probiotics and combinations thereof.

The invention is described below in more detail by means of the following examples, which are only given for purposes of illustration.

All references to percentages are percentages by weight unless stated otherwise.

### EXAMPLES

### Example I

A. Induction of Colitis by DNBS and Administration of Bacteria

The strain of Streptococcus thermophilus CNRZ160 according to the invention was first tested, and compared with another strain of S. thermophilus, PBSMJ, for its capacity to limit, in vivo, an intestinal inflammation in an individual.

A low-grade inflammation is induced in mice by injection of a low dose of DNBS.

The protocol of DNBS-induced colitis is carried out as described previously (Martin R, et al. Inflamm Bowel Dis. March 2014; 20(3): 417-30).

Briefly, the mice are anesthetized with isoflurane (Abbott, Abbott Park, Ill.) and a 10 cm long segment of PE-90 tube (ClayAdam, Parsippany, N.J.) is fixed to a tuberculin syringe and inserted to 3.5 cm in the colon.

Colitis is induced by intrarectal injection (i.r.) of 200 mg/kg of solution of DiNitroBenzene Sulfonic acid (DNBS) (ICN, Biomedical Inc.) in 30% ethanol (EtOH), via this tube.

The control mice (without colitis) only receive ethanol (EtOH).

The mice are fed with 6% sucrose in the drinking water for the first 3 days after injection of DNBS to prevent dehydration (DNBS period). 10 days after the DNBS period, 200 μl containing 1×10⁹ CFU of one of the bacterial strains discussed hereunder is administered by the intragastric route, every day for 10 days (gavage period).

The colitis is reactivated 21 days after the first injection of DNBS (recovery period) with a second injection of 100 mg/kg of DNBS solution.

The study groups are as follows: non-colitis control group (Ethanol+PBS), colitis control group (DNBS+PBS), group of S. thermophilus strain PBSMJ (DNBS+PBSMJ) and S. thermophilus CNRZ160 (DNBS+CNRZ160).

B. Measurement of Intestinal Permeability as Marker of Inflammation

Mice (n=8/group) were fed by gastric tube, or not, on the basis of the protocol indicated above, with the strains of S. thermophilus PBSMJ or CNRZ160, grown in milk.

An increase in intestinal permeability is then observed in these mice by assay of FITC in the blood.

In fact we observe a doubling of the concentration of FITC between the groups PBS ethanol (uninflamed control) and PBS DNBS (inflamed control), which reflects an increase in permeability.

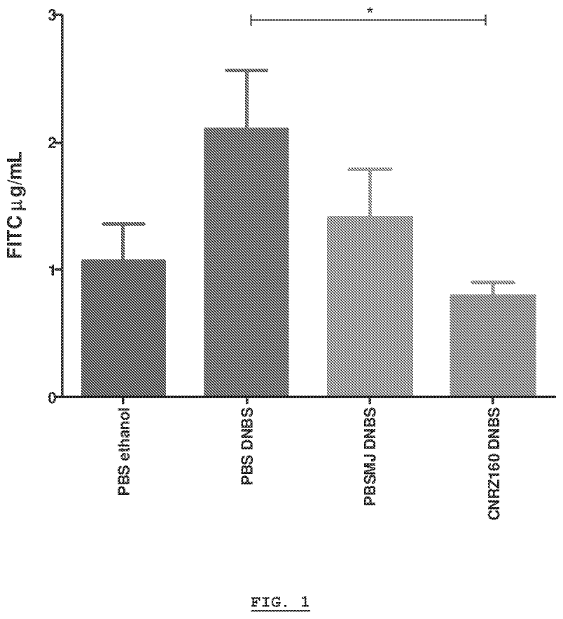

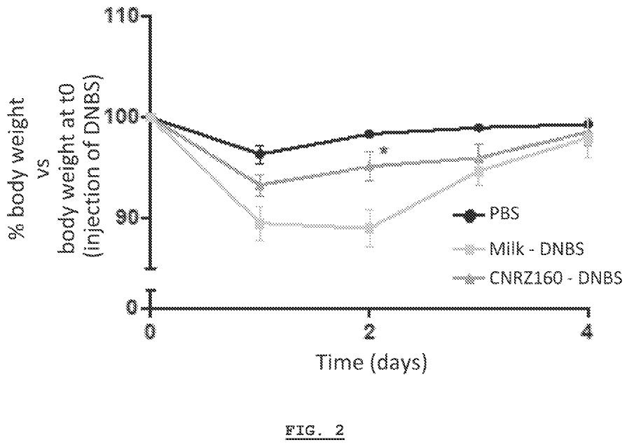

The results obtained are shown in FIG. 1.

Gavage with the CNRZ160 strain allows complete reestablishment of intestinal permeability.

Conversely, gavage with the PB5MJ strain does not have an effect on restoration of intestinal permeability.

Thus, a strain with reference CNRZ160 according to the invention effectively displays an anti-inflammatory effect.

### Example II—Measurement of the Effect of the Strain CNRZ160 on Weight Loss

Acute inflammation is induced in mice by injecting a high dose of DNBS.

The protocol for DNBS-induced colitis is carried out as described previously (Martin R, et al. FEMS Microbiology Review, 2017, S49-S70)).

Briefly, the mice are anesthetized with isoflurane (Virbac France Espace Azur Mercantour—3e rue—LID—06510 Carros) and a 10 cm long segment of PE-90 tube (ClayAdam, Parsippany, N.J.) is fixed to a tuberculin syringe and inserted to 3.5 cm in the colon.

Colitis is induced by intrarectal injection (i.r.) of 3000 mg/kg of solution of DiNitroBenzene Sulfonic acid (DNBS) (ICN, Biomedical Inc.) in ethanol at 30% (EtOH), via this tube.

The control mice (without colitis) only receive ethanol (EtOH).

Six days before the DNBS period, 200 μl containing 1×10⁹ CFU of one of the bacterial strains discussed hereunder is administered by the intragastric route, every day for 10 days (gavage period). The DNBS is administered at t0 and the mice are sacrificed 4 days after injection of DNBS.

DNBS mice were prepared and fed by gastric tube with the reference strain CNRZ160 grown in milk.

The weight of these mice (n=8/group) is then measured every day for 4 days.

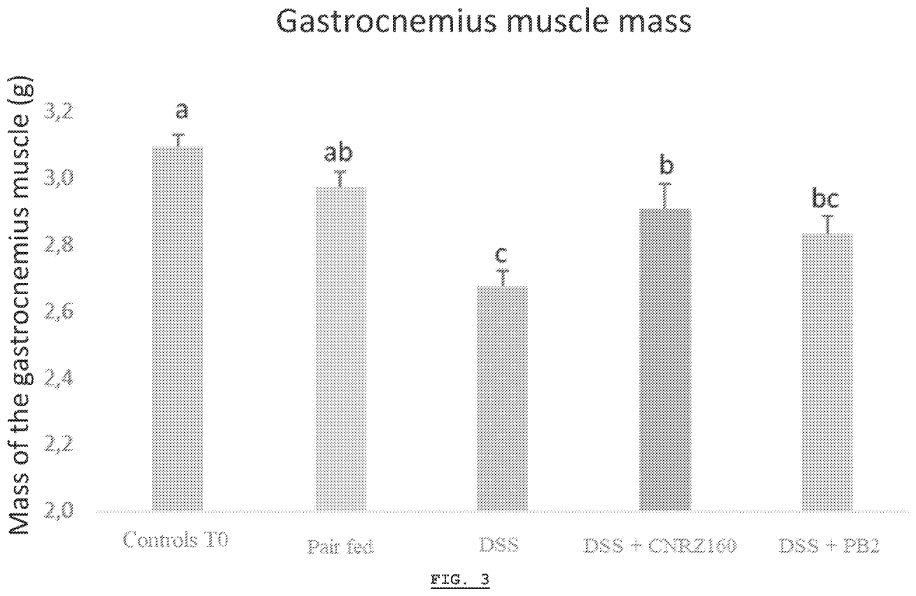

The results obtained are shown in FIG. 2.

The Milk-DNBS mice (inflamed control) had lost up to 10% of their weight after 2 days (D2) and then recovered a normal weight corresponding to that of the PBS group (uninflamed control).

The mice fed by gastric tube with CNRZ160 lose less weight than the inflamed control group. The difference is significant at D2 relative to the Milk-DNBS group.

### Example III

The strain of Streptococcus thermophilus CNRZ160 according to the invention was also tested, and compared with another strain of S. thermophilus, PB5MJ, in another model of intestinal inflammation, for its capacity to:
limit in vivo muscle wasting of animals with an intestinal inflammation.
limit the deaths of said individuals.
limit the loss of lean mass.
limit hypertrophy of the colon.

The various protocols and results obtained in this direction are presented hereunder.

A. Induction of Colitis by DSS and Administration of Bacteria

To induce colitis, drinking water supplemented with 2% (w/v) of dextran sodium sulfate (DSS; MP Biomedicals, LLC, Aurora, Ohio, USA) is administered to the mice for 7 days. The mice then recover for 5 days by drinking water without supplement (FIG. 7a). 6-Formylindolo[3,2-b]carbazole (Ficz) obtained from Enzo Life Sciences (Lausanne, Switzerland), and resuspended in dimethyl sulfoxide (DMSO; Sigma-Aldrich), is administered intraperitoneally 1 day after administration of DSS (1 μg/mouse).

The controls consist of mice injected with DMSO only.

B. Tests for Loss of Muscle Mass

a. As indicated above, the inventors studied the effect of ingestion of S. thermophilus CNRZ160 on muscle wasting during intestinal inflammation generated chemically by the ingestion of dextran sodium sulfate (4% DSS in the drinking water) over a period of 28 days.

The experimental groups compared are as follows:
a group of control animals euthanized at the start of the experiments to measure the weight of the muscles before treatment with DSS (Controls T0 group);
a group of control animals treated with DSS without administration of S. thermophilus according to the invention (group DSS);
a group of animals treated with DSS and supplemented with Streptococcus thermophilus CNRZ160, called group “DSS+CNRZ160”; and
a comparative group of animals treated with DSS and supplemented with Streptococcus thermophilus PB5MJ, called group “DSS+PB2”.
a group of pair-fed animals of the group DSS, called PF group or Pair Fed group, insofar as ingestion of DSS is reflected in a slight decrease in amount ingested by the animals, which has an effect per se on muscle mass. Thus, the animals in group PF are not treated with DSS but only receive a daily amount of food equal to that ingested by the animals in all the groups DSS with or without the bacterial strains.

The amounts of feedstuffs ingested by the animals in the different groups DSS, PF, DSS+CNRZ160 and DSS+PB2 are identical.

Moreover, the effects of the experimental treatments on the weight of the animals are monitored using a balance.

b. Results

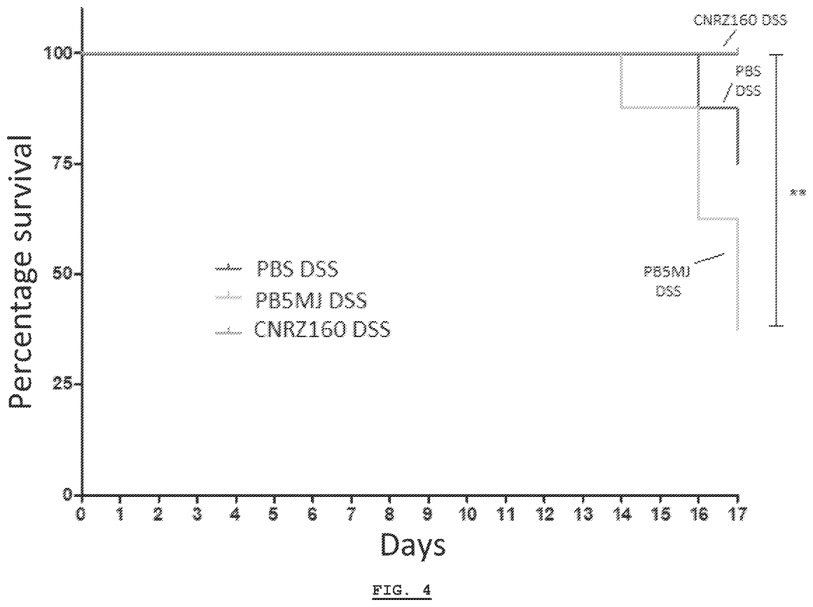

Pair-feeding, corresponding to an amount ingested less than ad libitum, is reflected in a weight loss of the animals, as illustrated in FIG. 3. This weight loss is accentuated with ingestion of DSS.

The effect of the various experimental treatments on the weight of the muscles of the animals, and in particular on the weight of the gastrocnemius muscle, is also shown in FIG. 3.

It can thus be seen that at the end of the 28 days of treatment with DSS, the animals supplemented with S. thermophilus CNRZ160 (group DSS+CNRZ160) have a statistically greater muscle mass than the DSS group of animals, which is not the case for the group of animals supplemented with S. thermophilus PB5MJ (group DSS+PB2).

The mass of the animals of group DSS+CNRZ160 is statistically similar to that of the animals of group PF.

It is thus demonstrated that supplementation of the animals with the bacterium S. thermophilus CNRZ160 makes it possible to limit muscle wasting of the animals during the experimental period of treatment with DSS, and that not all the strains of S. thermophilus possess such properties, as demonstrated with the PB5MJ strain.

C. Tests for Mortality

a. The aforementioned mice treated with DSS were, prior to induction of colitis, fed by gastric tube for 5 days with the CNRZ160 strain or the PB5MJ strain.

A survival curve was measured for each of the different populations of mice: a group of mice (n=8) treated with DSS but only treated previously with PBS (control—PBS DSS), a group of mice (n=8) treated with DSS and fed previously by gastric tube with CNRZ160 (CNRZ160 DSS) and a group of mice (n=8) treated with DSS and fed previously by gastric tube with PB5MJ (PB5MJ DSS).

b. Results

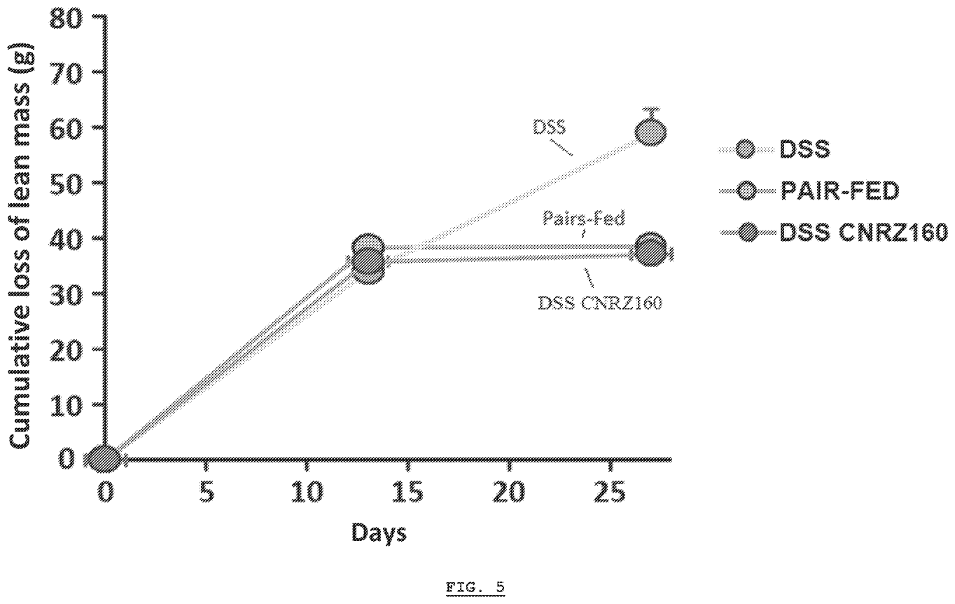

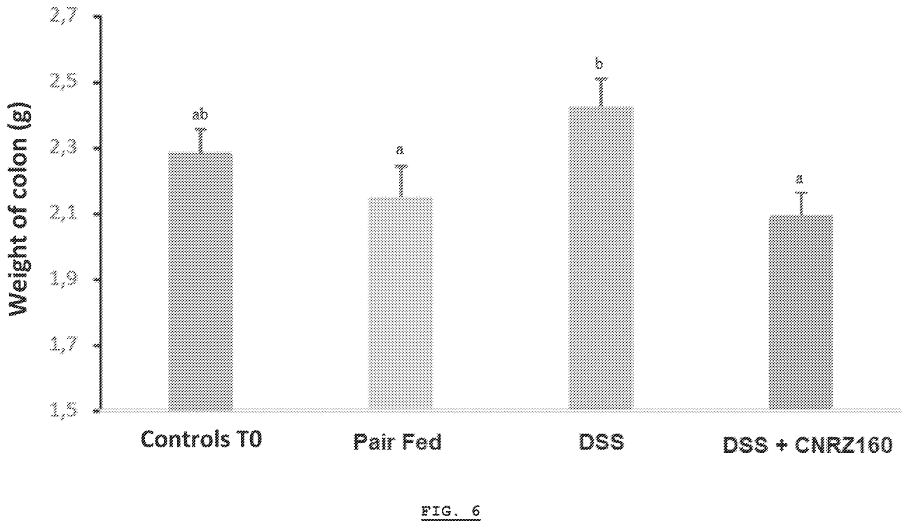

The results obtained are shown in FIG. 4.

Treatment with DSS leads to death of 25% of the mice in the untreated control group (PBS DSS).

A higher mortality is observed in the group fed previously by stomach tube with PB5MJ, namely of the order of 60%.

Conversely, no mortality is observed in the group of mice fed previously by stomach tube with CNRZ160.

D. Tests for Loss of Lean Mass

a. Rats as mentioned above treated with DSS were, prior to induction of colitis, fed by gastric tube with the CNRZ160 strain.

A curve representing the cumulative loss of lean mass was constructed for each of the different populations of rats tested, namely:
a group of rats (n=12) treated with DSS but only treated previously with PBS (control—DSS),
a group of rats (n=12) treated with DSS and fed previously by gastric tube with CNRZ160 (DSS CNRZ160), and
a group of rats (n=12) pair-fed in the group DSS, not treated with DSS but only receiving a daily amount of food equal to that ingested by the animals in the group DSS.

The lean mass is measured using the EchoMRI-700.

The EchoMRI-700 is a quantitative nuclear magnetic resonance system which makes it possible to obtain precise measurements of the parameters of body composition: total fat body mass, total lean body mass, free water and total body water in rats or mice.

Taking measurements does not require anesthesia or sedation.

Set-up and calibration of the equipment are automatic.

The radiation is nonionizing and very reproducible.

b. Results

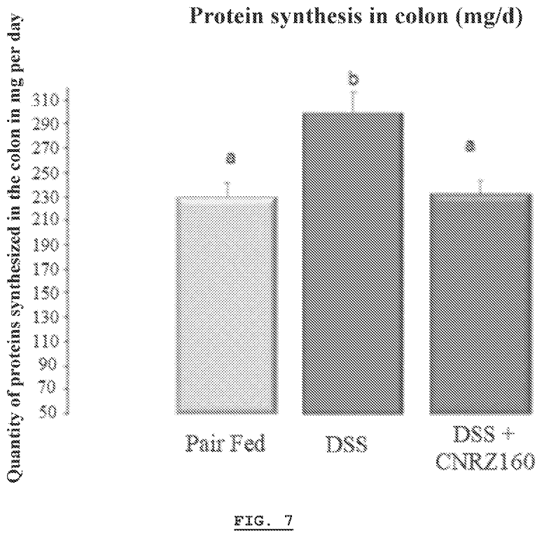

The results obtained are shown in FIG. 5.

The loss of lean mass was measured over 25 days counting from the treatment with DSS.

A constant decrease in lean mass is observed throughout the duration of the measurements, the animals in group DSS losing about 60 g of lean mass at the end of the 25 days of observations.

Conversely, the loss of lean mass observed in the group treated with the CNRZ160 strain is identical to that observed in the Pair-Fed population, namely about 40 g at the end of 25 days of observation, which is well below the 60 g of losses observed in the group DSS. More particularly, we observe stoppage, or at the very least considerable slowing, of the loss of lean mass starting from 12 days in the groups Pair-Fed and DSS CNRZ160.

E. Tests for Limitation of Hypertrophy of the Colon

a. Rats as mentioned above treated with DSS were, prior to induction of colitis, fed by gastric tube with the CNRZ160 strain.

The colon of each animal was isolated and cautiously removed and weighed to evaluate its mass.

More particularly, the mass of the colon of different populations of rats was measured after 28 days of administration of DSS:
a group of control animals (n=12) euthanized at the start of the experiments to measure the weight of the colon before treatment with DSS (group Controls T0);
a group of rats (n=12) pair-fed in the group DSS, not treated with DSS but only receiving a daily amount of food equal to that ingested by the animals in the group DSS,
a group of rats (n=12) treated with DSS but only treated previously with PBS (control—DSS), and
a group of rats (n=12) treated with DSS and fed previously by gastric tube with CNRZ160 (DSS CNRZ160).

b. Results

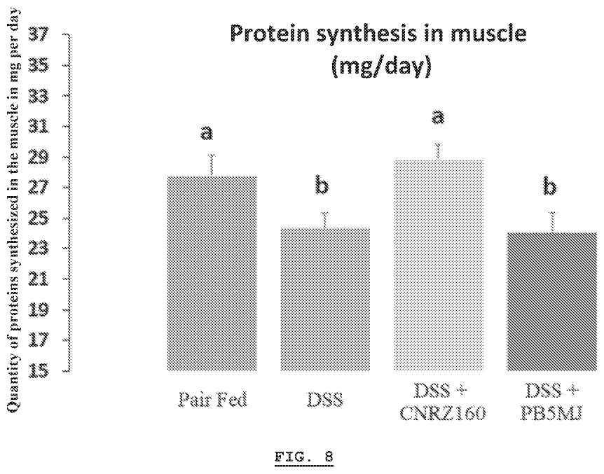

The results obtained are shown in FIG. 6.

Hypertrophy of the colon is observed in the group DSS, with a significant increase in the mass of the colon in this population of individuals compared to the groups Controls T0.

Conversely, a slight decrease in the mass of the colon is observed in the Pair Fed population.

Moreover, the population of individuals treated both with DSS and with the CNRZ160 strain have a colon with mass similar to that of the Pair Fed group. Thus, it appears that treatment with the CNRZ160 strain makes it possible, advantageously, to reduce the effects of DSS inducing hypertrophy of the colon when the latter is coadministered with the DSS for 28 days.

F. Tests for Protein Synthesis of the Colon and Gastrocnemius Muscle

a. The protein synthesis of the colon or gastrocnemius muscle of different populations of rats was measured after 28 days of administration of DSS.

More particularly, protein synthesis at the level of the colon of the following different population groups were measured after 28 days of administration of DSS:
a group of rats (n=12) pair-fed in the group DSS, not treated with DSS but only receiving a daily amount of food equal to that ingested by the animals in the group DSS,
a group of rats (n=12) treated with DSS but only having been treated with PBS (control—DSS), and
a group of rats (n=12) treated with DSS and treated simultaneously with the bacterium CNRZ160 (DSS CNRZ160).

Regarding protein synthesis at the level of the gastrocnemius muscle, the following different population groups were measured after 28 days of administration of DSS:
a group of rats (n=12) pair-fed in the group DSS, not treated with DSS but only receiving a daily amount of food equal to that ingested by the animals in the group DSS,
a group of rats (n=12) treated with DSS but only having been treated with PBS (control—DSS),
a group of rats (n=12) treated with DSS and treated simultaneously with the bacterium CNRZ160 (DSS+CNRZ160), and
a group of rats (n=12) treated with DSS and treated simultaneously with the bacterium PB5MJ (DSS+PB5MJ).

For measurement of protein synthesis, the high dose technique is used.

150 μmol/100 g (100% Valine 1−¹³C) is injected intravenously 20 minutes before the animal is sacrificed.

Measurement of the plasma, intra-tissue and intra-proteins enrichment (Valine 1-¹³C) of the gastrocnemius muscle or colon makes it possible to determine a rate of incorporation of amino acids in the proteins and therefore to give a value of protein synthesis.

The details of the procedure for sample preparation, measurement of enrichment with 13C valine and calculation are detailed in Jarzaguet et al. 2018 (Food Funct. Dec. 13, 2018; 9(12): 6526-6534).

b. Results

The results obtained for protein synthesis at the level of the colon are shown in FIG. 7. The results obtained for protein synthesis at the level of the gastrocnemius muscle are shown in FIG. 8.

Thus, firstly, at the level of the colon, we observe a return of protein synthesis of the colon to normal during treatment with the CNRZ160 strain.

Similarly, we observe maintenance of protein synthesis at the level of the gastrocnemius muscle in the populations treated with the CNRZ160 strain. However, this is not the case in the individuals treated with the PBSMJ strain.

G. Statistical Analysis

Statistical analysis is carried out by means of the Sigma Plot software SigmaPlot 12, Systat software, San Jose, USA). All the data are expressed in the form of mean value+/−SEM.

The comparisons are carried out depending on the case by 1-factor ANOVA followed by a post hoc LSD Fisher test. A value p below 0.05 is considered to be significant.

1. Method for preventing and/or treating an inflammatory intestinal disorder and/or a disorder caused by said inflammatory intestinal disorder in an individual, comprising administering to said individual bacterial strain of the species Streptococcus thermophilus deposited with the CNCM under accession number CNCM 1-5334.

2. The method as claimed in claim 1, wherein said bacterial strain is used in a live, semi-active, inactivated or dead form.

3. The method as claimed in claim 1, wherein the individual is a mammal.

4. The method according to claim 1, wherein the disorder caused by said inflammatory intestinal disorder is selected from the group consisting of weight loss, loss of lean mass, muscle loss, hypertrophy of the colon, a change in intestinal permeability and any combination of these disorders.

5. The method according to claim 1, wherein the disorder caused by the inflammatory intestinal disorder is loss of lean mass and of muscle mass.

6. The method according to claim 1, wherein the inflammatory intestinal disorder is a chronic inflammatory disease of the intestine.

7. The method according to claim 1, wherein the bacterial strain is included in a composition comprising a physiologically acceptable medium.

8. The method according to claim 1, wherein the composition is suitable for administration of a daily dose representing from 10⁷ to 10¹¹ colony forming units (CFU).

9. The method according to claim 8, wherein the composition is for administration by the oral route and is selected from the group consisting of a food product, a drink, a pharmaceutical product, a nutraceutical, a food additive, a food supplement and a milk product.

10. The method according to claim 6, wherein the chronic inflammatory disease of the intestine is selected from the group consisting of Crohn's disease, hemorrhagic rectocolitis and pouchitis.

11. The method according to claim 7, wherein the bacterial strain is included in an oral composition comprising a physiologically acceptable medium.

12. The method according to claim 9, wherein the composition is a milk product or a food supplement.
